(19) United States
(12) Patent Application Publication
(10) Pub. No.: US 2007/0276395 A1
(43) Pub. Date: Nov. 29, 2007

(54) Surgical Drill

(75) Inventor: Peter James Burn, Christchurch (NZ)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: ENZTEC LIMITED, Christchurch (NZ)

(21) Appl. No.: 11/632,298

(22) PCT Filed: Jul. 15, 2005
(86) PCT No.: PCT/NZ05/00173

§ 371 Date: Jan. 12, 2007

(30) Foreign Application Priority Data
Jul. 20, 2004 (NZ) 534215

(51) Int. Cl. A61B 17/16

(52) U.S. Cl. 606/80

(57) ABSTRACT

A surgical drill which includes a drill tip (3) rigidly secured to a shaft (2) for attachment of the drill to a rotary driving means, the end of the drill tip remote from the shaft being formed with cutting edges (4,5) tapering to a point (6), and the drill tip (3) having a cross-sectional shape such that the non-tapered portion of the drill tip (3) is substantially wider in a first direction (w) than in a second direction (x); the shaft (2) having a width at its widest point adjacent the drill tip (3) which is substantially smaller than the maximum cross-sectional width of the drill tip (3); the drill tip (3) being formed with at least one notch (8) for picking up a suture.

### TECHNICAL FIELD

The present invention relates to a surgical drill for use in any application where an aperture needs to be made through a bone and a suture pulled through the aperture e.g. for the tightening/repair of ligaments after total knee replacement, or for ligament avulsion following trauma to the knee or for soft tissue reattachment in total tip arthroplasty, or for general ligament/tendon reattachment to bone.

### BACKGROUND ART

At present, the technique used it to drill a hole through the bone with a conventional drill, then withdraw the drill from the bone and push a needle or a seeker through the hole, thread the suture through the needle eye and pull the suture back through the hole. The suture is then secured (e.g. by knotting it or by securing it to a securing button) to prevent it from pulling back through the hole. The multiple steps required are time-consuming, and threading the suture through the needle eye is difficult.

### DISCLOSURE OF INVENTION

An object of the present invention is to provide any improved surgical drill which enables the hole to be drilled and the suture picked up and drawn through the hole in a single sequence, without withdrawing the drill.

The present invention provides a surgical drill which includes a drill tip rigidly secured to a shaft for attachment of the drill to a rotary driving means, the end of the drill tip remote from the shaft being formed with cutting edges tapering to a point, and the drill tip having a cross-sectional shape such that the non-tapered portion of the drill tip is substantially wider in a first direction than in a second direction; the shaft having a width at its widest point adjacent the drill tip which is substantially smaller than the maximum cross-sectional width of the drill tip; the drill tip being formed with at least one notch for picking up a suture.

Preferably, the drill tip and the drill shaft are formed integrally. Preferably also, the drill tip is rectangular in cross-section.

Preferably, the drill tip also includes more than one notch and/or one or more eyes through which a suture may be threaded.

Preferably also, the drill shaft is marked adjacent the end of the shaft remote from the drill tip to indicate the orientation of the flattened drill tip, so that the orientation of the drill tip and the position of the notch can be determined even when the drill tip itself is out of sight.

The drill of the present invention may be made of any suitable tough material which is approved for medical use and which is capable of holding a cutting edge.

### BRIEF DESCRIPTION OF THE DRAWINGS

By way of example only, a preferred embodiment of the present invention is described in detail, with reference to the accompanying drawings, in which:—

FIG. 1 shows a side view of part of the drill of the present invention;

FIG. 2 shows the drill of FIG. 1 rotated through 90°;

FIG. 3 is a section on line A-A of FIG. 1; and

FIG. 4a-c are side views of the drill tip, showing different notches.

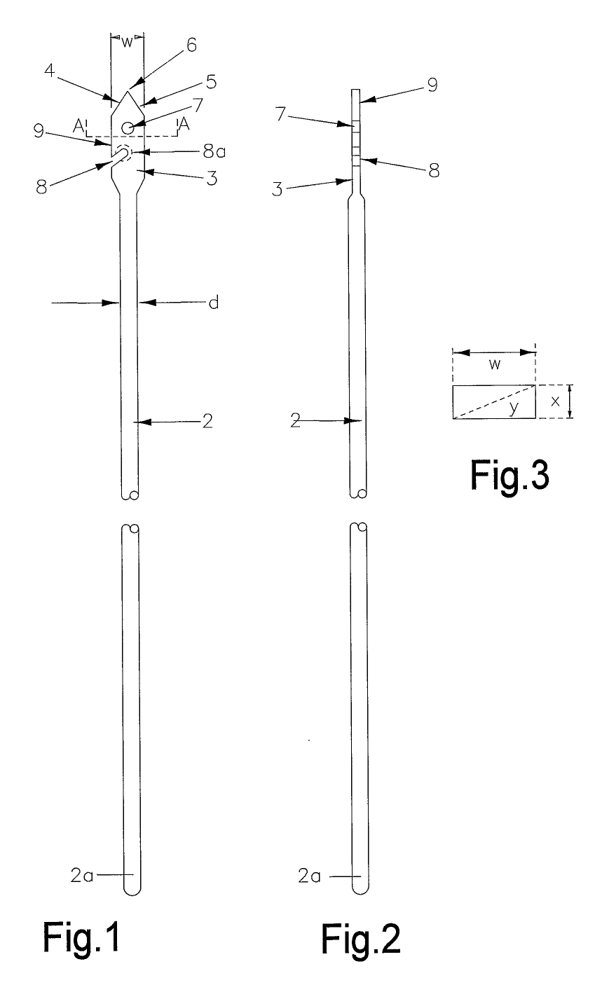

### BEST MODE FOR CARRYING OUT THE INVENTION

Referring to the drawings, a drill in accordance with the present invention comprises a drill shaft 2 formed integrally with a drill tip 3. The shaft and tip are formed of a suitable material e.g. surgical steel. The drill shaft 2 is circular in cross-section; typical diameters would be in the range 1.0-3.0 millimetres. The end 2a of the drill shaft 2 remote from the drill tip 3 in use is connected to the chuck of any of a range of known rotary drives, to allow the drill to be rotated in known manner.

As shown in FIG. 3, the non-tapered portion of the drill tip 3 has a cross-sectional shape which is substantially wider in a first direction w than in a second direction x. In the particular example shown in the drawings, the cross-sectional shape is rectangular, but it will be appreciated that a variety of different cross-sectional shapes could be used, providing they met the above criterion. The reason for the cross-sectional shape is to allow a suture to be passed through the hole formed by the drill, without having to withdraw the drill from the hole and without the risk of the drill fouling the suture. Further, the maximum width w of the drill tip is substantially greater than the diameter d of the drill shaft adjacent the tip, although of course the shaft may be enlarged at its end 2a remote from the tip, for connection to the chuck of a known rotary drive, or fitted to a quick-coupling drive. A collar (not shown) may be provided around the driving end to allow manual manipulation and orientation. The collar may be marked to indicate the orientation of the greatest width of the tip 3 and the side of the tip 3 carrying the notch. Alternatively, the shaft 2 may be marked adjacent the end of the shaft remote from the tip 3 to indicate orientation.

The end of the drill tip remote from the shaft is formed with a pair of opposed cutting edges 4,5 tapering to a point 6. A short distance back from the point 6, a hole 7 is formed through the tip, and a notch 8 its formed in one of the sides 9 of the tip having the smallest width; the notch 8 opens into the side 9 of the tip and is inclined towards the point 6. The edges of the hole 7 and notch 8 are smooth and rounded so that they do not damage sutures or tissues. The notch 8 may be enlarged at its inner end, as indicated in broken lines 8a. The inclination of the notch may be varied as required, to suit particular applications.

The above described drill is used as follows: —the end 2a of the shaft remote from the tip 3 is connected to a rotary drive of known type, and the drill is used to drill a hole through a selected position on a bone, with the point 6 of the drill in the centre of the hole. Since the tip 3 is rotated about the point 6, the hole cut through the bone by the cutting edges 4,5 is circular and has a final diameter equal to the diagonal y of the drill tip, i.e. slightly greater than the maximum width w of the drill tip. This means that once the hole is cut the drill may be extended further through the hole without the shaft 2 enlarging or damaging the hole.

When the hole has been cut, a suture may be threaded through the eye 7 in known manner, or the notch 8 may be used to pick up the suture and draw it back through the hole. If necessary, the drill may be turned through 90° and reinserted through the hole to collect a second or subsequent suture, without any risk of damage to sutures already drawn through the hole, because the drill tip is substantially wider in the direction w than in the direction x, so that there is plenty of clearance between the edge of the hole and the width of the drill tip in the direction x. Similarly, sutures can be passed in either direction through the hole whilst the drill is in place, without risk of damage to the sutures. The sutures are secured in any suitable manner to prevent them from pulling back through the hole.

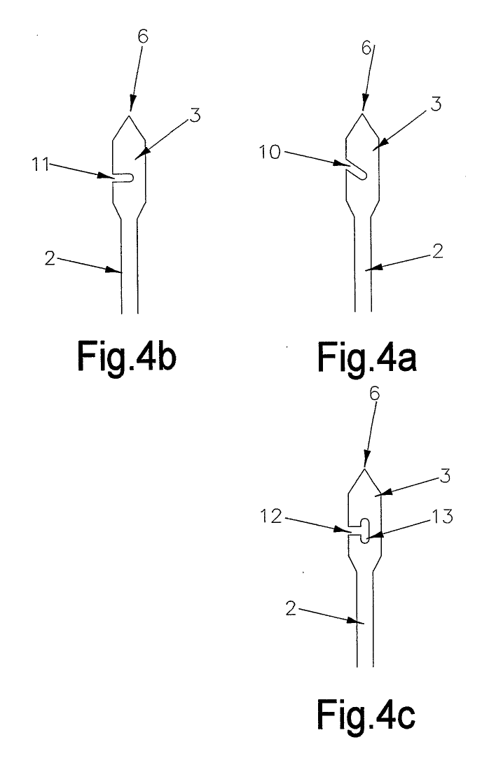

It will be appreciated that the dimensions and proportions of the drill shaft/drill tip and the orientation and position of the notch 8 may be varied as necessary to suit particular applications. FIGS. 4a-4c show three possible variations of the notch:—in FIG. 4a, the notch 10 is in the side of the drill tip 3, with the longitudinal axis of the notch inclined away from the point 6. In the variant shown in FIG. 4b, a notch 11 is formed in the side of the drill tip 3, with the longitudinal axis of the notch perpendicular to the longitudinal axis of the drill. In the variant shown in FIG. 4c, a notch 12 is formed in the side of the drill tip 3, with the longitudinal axis of the notch perpendicular to the longitudinal axis of the drill, but with a keyhole shaped portion 13 formed at the inner end of the notch. The keyhole shaped portion 13 provides a secured engagement between the walls of the notch and a suture, whether the suture is being pulled or pushed. More than one hole 7 and more than one notch 8 may be formed on the tip 3 if required.

To prevent wear of the drill causing eventual failure, it is preferred that the drill is single-use only.

1. A surgical drill which includes a drill tip rigidly secured to a shaft for attachment of the drill to a rotary driving means, the end of the drill tip remote from the shaft being formed with cutting edges tapering to a point, and the drill tip having a cross-sectional shape such that the non-tapered portion of the drill tip is substantially wider in a first direction than in a second direction; the shaft having a width at its widest point adjacent the drill tip which is substantially smaller than the maximum cross-sectional width of the drill tip; the drill tip being formed with at least one notch for picking up a suture.

2. The surgical drill as claimed in claim 1, wherein the drill tip and the drill shaft of formed integrally.

3. The surgical drill as claimed in claim 1, wherein the or each notch is formed on a side of the drill tip.

4. The surgical drill as claimed in claim 1, wherein the drill tip further includes at least one eye through which a suture may be threaded.

5. The surgical drill as claimed in claim 1 wherein the drill tip is rectangular in cross-section.

6. The surgical drill as claimed in claim 1 wherein the drill shaft is marked adjacent the end of the shaft remote from the drill tip, to indicate the orientation of the drill tip.

7. The surgical drill as claimed in claim 1 further including a collar adjacent the end of the shaft remote from the drill tip, said collar being marked to indicate the orientation of the drill tip.

8. The surgical drill as claimed in claim 1 wherein the drill tip is rectangular in cross section and includes at least one eye through which the suture may be threaded.
